(19) United States
(12) Patent Application Publication
(10) Pub. No.: US 2009/0054860 A1
(43) Pub. Date: Feb. 26, 2009

(54) Composite Fabric Panel For Use In Disposable Absorbent Articles

(76) Inventors: Terrill A. Young, Cincinnati, OH (US); Donald C. Roe, West Chester, OH (US); William J. Toerner, Wyoming, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/261,270

(22) Filed: Oct. 30, 2008

Related U.S. Application Data
(62) Division of application No. 09/566,747, filed on May 9, 2000, now abandoned.

(51) Int. Cl. A61F 13/15; B32B 3/20; A61F 13/535

(52) U.S. Cl. 604/360; 428/305.5; 428/36.1; 428/297.4; 428/174; 428/186; 428/300.7; 428/99; 604/385.23; 604/386; 604/361

(57) ABSTRACT

A composite fabric panel comprising a substrate and a plurality of elongate operative members engaged directly with the substrate, wherein the elongate operative members perform a serviceable operation in response to a stimulus. In one embodiment, the substrate comprises a nonwoven and the elongate operative members include multifilament yarns. The serviceable operation performed by the elongate operative members may comprise a transformation and/or a selective dispersion of active agents in response to such stimuli as wetting, temperature change, change in pH or affects of concentrated chemical environment.

### CROSS REFERENCE TO RELATED APPLICATION

This application is a divisional application of U.S. application Ser. No. 09/566,747, filed May 9, 2000, which is hereby incorporated by reference in its entirety.

### FIELD OF THE INVENTION

The present invention relates to composite fabric panels, particularly to a composite fabric panel configured for use in disposable absorbent articles.

### BACKGROUND OF THE INVENTION

Today, disposable articles are widely used for treatment of living and non-living surfaces for a wide variety of purposes. For example and without limitation, disposable products are used in the areas of personal care items, pharmaceuticals and health care, baby care, textile care, and home, commercial and industrial cleaning. These disposable products may be used for purposes such as, but not limited to cleaning, applying coatings or other materials to treat surfaces (such as stains, paints, waxes, conditioning agents, etc.), and containing or removing materials such as wastes, contaminants or other excess or undesired materials (such as diapers for body wastes, mops, brooms, gloves, etc. In many cases these articles have replaced reusable articles as the preferred means for reasons such as cost, convenience, and sanitation.

While many advancements have been made in the field of disposable articles to improve their effectiveness, such as improved strength, addition of active ingredients to improve cleaning, use of absorbent gellants to improve absorptivity, etc., a number of problems still exist. Among the problems experienced with these disposable treatment articles are leakage of materials intended to be removed or isolated, which can lead to incomplete cleaning and contamination of the surface that was treated by the material that was intended to be removed or isolated by the article, etc.

Another problem is that disposable treatment articles to date do not discriminate between circumstances when a particular type of treatment is needed and when it is not. For example, a disposable wipe may be used for general cleaning as well as for cleaning biological contaminants (such as wiping away feces from surface of a baby's skin). Wipes designed to remove feces from a baby's buttocks contain disinfectants or other active ingredients that may not be needed, or is undesired, for other uses, such as wiping food from the baby's face, or wiping stains from a textile surface or floor. Further, in the event that the disposable article is to be used multiple times before disposal, an active ingredient needed for treating a particular condition, such as disinfection of the skin in association with removal of feces, may no longer be present or effective for its intended purpose.

Accordingly, it would be desirable to provide a composite fabric panel for use in disposable absorbent articles that can effectively absorb and store bodily waste while exhibiting effective functional characteristics. It would also be desirable to provide a composite fabric panel having improved aesthetics. Further, it would be desirable to provide such a composite fabric panel capable of performing serviceable operations such as delivering active agents in response to a stimulus.

### SUMMARY OF THE INVENTION

A composite fabric panel comprising a substrate and a plurality of elongate operative members engaged directly with the substrate, wherein the elongate operative members perform a serviceable operation in response to a stimulus. The serviceable operation can include absorption as well as the delivery of active agents in response to such stimuli as wetting or detecting a particular constituent. For one embodiment, the substrate includes a pair of overlapping nonwoven fabric layers and the elongate operative members are enclosed within the substrate between the layers delineating elongated sections of the layers between pairs of adjacent elongate operative members. The elongated sections of the layers converge to adjoining portions that are spaced transversely from the elongate operative members enclosing channels that extend alongside the elongate operative members.

The serviceable operation performed by the elongate operative members may comprise a transformation such as a change in color, expansion/contraction, or odor absorption occurring in response to such stimuli as wetting, temperature change, change in pH or affects of a concentrated chemical environment. Alternatively, the serviceable operation may comprise the selective dispersion of an active agent in response to the stimuli. The active agent can comprise an anti-microbial, an enzyme, a pH neutralizer, or a medication.

In a preferred embodiment, the substrate comprises a nonwoven sheet and the elongate operative members comprise yarns which are enclosed between overlapping layers of the nonwoven fabric sheet. In another embodiment, the elongate operative members are mounted on an outer side surface of the nonwoven sheet. Yet another embodiment comprises embedding the elongate operative members within the nonwoven sheet before bonding the nonwoven fibers together to place the sheet in a cohesive condition. For this embodiment, the elongate operative members can comprise filaments formed of heat shrinkable material bonded to the substrate which deflect the substrate upon shrinkage of the filaments under the influence of heat.

The composite fabric panel may be included as a component of an absorbent article such as a disposable diaper or the like. For this embodiment, the substrate has an elongated configuration with a crotch region located between a pair of opposite end regions. The elongate operative members extend from the crotch region of the substrate into the end regions. The elongate operative members can be made to absorb bodily fluid discharged in the crotch region and draw it into the end regions. In a preferred embodiment, the substrate comprises a nonwoven sheet forming a topsheet component of the disposable diaper. In another preferred embodiment, the substrate comprises a liquid impervious film forming a backsheet component of the disposable diaper.

### BRIEF DESCRIPTION OF THE DRAWINGS

While the specification concludes with claims particularly pointing out and distinctly claiming the subject matter which is regarded as forming the present invention, it is believed that the invention will be better understood from the following description which is taken in conjunction with the accompanying drawings in which like designations are used to designate substantially identical elements, and in which:

FIGS. 1a, and 1b are top views of the composite fabric panel of the present invention showing the elongate operative members disposed unidirectionally along parallel linear paths.

FIG. 1c is a top view of the composite fabric panel of the present invention showing the elongate operative members disposed unidirectionally along parallel nonlinear (sinusoidal) paths.

FIG. 2 is a top view of the composite fabric panel of the present invention showing the elongate operative members disposed bi-directionally forming an intersecting pattern.

FIG. 3 is a sectional view of the composite fabric panel shown in FIG. 1;

FIG. 4 is an enlarged partial view of parts shown in FIG. 3;

FIG. 5 is a schematic view of parts of an apparatus used to construct the composite fabric panel shown in FIG. 1;

FIGS. 6 and 7 are views similar to FIG. 3 showing alternative composite fabric panels constructed in accordance with the invention;

FIGS. 8A, 8B and 8C are schematic views of differing arrays of yarns used in accordance with the invention;

FIG. 9 also is a view similar to FIG. 3 showing an alternative composite fabric panel constructed in accordance with the invention;

FIG. 10 is a schematic view indicating steps taken in an alternative method of constructing a composite fabric panel in accordance with the invention;

FIGS. 11 and 12 are additional views similar to FIG. 3 showing alternative composite fabric panels constructed in accordance with the invention;

FIG. 13 is a top view of an absorbent article comprising a preferred embodiment of the present invention;

FIG. 14 is an enlarged partial view of a composite fabric panel included as a component of the absorbent article of FIG. 13;

FIG. 15 is a top view of a diaper topsheet constructed as a composite fabric panel in accordance with the invention;

FIG. 16 is a top view of an alternative diaper topsheet constructed in accordance with the invention; and

FIG. 17 is a view similar to FIG. 13 showing an absorbent article comprising an additional embodiment of the invention, with this view being taken from an opposite side of the absorbent article.

### DETAILED DESCRIPTION OF THE INVENTION

While this specification concludes with claims particularly pointing out and distinctly claiming that which is regarded as the invention, it is anticipated that the invention can be more readily understood through reading the following detailed description of the invention and study of the included drawings.

### DEFINITIONS

As used herein, the term “absorbent article” refers to devices which absorb and contain liquid, and more specifically, refers to devices used for cleaning, such as wipes, as well as articles which are placed against or in proximity to the body of the wearer to absorb and contain the various exudates discharged from the body.

“Longitudinal” is a direction running parallel to the maximum linear dimension of the article. Longitudinally includes directions within ±45° of the longitudinal direction.

The “lateral” or “transverse” direction is orthogonal to the longitudinal direction.

The “Z-direction” is orthogonal to both the longitudinal and transverse directions.

The term “disposable” is used herein to describe absorbent articles which generally are not intended to be laundered or otherwise restored or reused as an absorbent article (i.e., they are intended to be discarded after a single use and, preferably, to be recycled, composted or otherwise disposed of in an environmentally compatible manner).

As used herein, the term “disposed” is used to mean that an element(s) is formed (joined and positioned) in a particular place or position as a unitary structure with other elements or as a separate element joined to another element.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

A “unitary” absorbent article refers to absorbent articles which are formed of separate parts united together to form a coordinated entity so that they do not require separate manipulative parts like a separate holder and liner.

As used herein, the term “diaper” refers to an absorbent article generally worn by infants and incontinent persons about the lower torso.

As used herein “fluid” refers to matter in the liquid state (e.g. bodily waste, moisture, oil etc.).

An “elongate operative member” is a slender object displaying functional characteristics such as physical transformation, absorption of fluids or dispersion of active agents.

A “stimulus” is something capable of influencing the activity of an object.

By “slender” it is meant that the object has a major axis which is very long compared to the two orthogonal axes and has an aspect ratio of at least 10/1, preferably at least 50/1.

A “nonwoven” is a manufactured sheet, web, or batt of directionally or randomly oriented natural or man-made fibers bonded by friction, and or cohesion, excluding paper and products which are woven, knitted, tufted, or felted by wet milling, whether or not additionally needled.

The composite fabric panel of the present invention is applicable in any field of use including without limitation, personal care, health care, baby care, home and household care, cleaning products, disinfecting and/or sanitizing products, textile treatment, personal care, cosmetics, medical care and pharmaceuticals, etc. Although the composite fabric panel can be useful in any of the aforementioned fields of use, it is particularly applicable to absorbent articles including without limitation, disposable diapers, incontinence briefs, incontinence undergarments, absorbent inserts, diaper holders and liners, feminine hygiene garments, wipes, mops, bandages, bibs and the like.

The composite fabric panel hereof comprises a planar substrate combined with elongate operative members which perform serviceable operations in response to stimuli that accentuate the properties and corresponding usefulness of the composite fabric panel. The substrate forms the body of the composite fabric panel and can generally comprise any single or multiple layer structure which may be composed of films, cellulosic webs, or nonwovens. The elongate operative members may include any number of different structures having elastic or inelastic characteristics and may be constructed from materials including, but not limited to, filaments, foams, tapes, yarns and the like. The elongate operative members are engaged directly with the substrate and may be joined thereto or embedded therein as further described hereunder.

In a preferred embodiment, the planar substrates include non-woven or woven fibrous or non-fibrous materials having elongate operative members comprising multifilament yarns incorporated into or onto the substrate. Such planar fibrous substrates may be disposable and include natural fibers, synthetic fibers, or mixtures of natural and synthetic fibers. Suitable natural fibers include but are not limited to cellulosic fibers, such as wood pulp fibers, cotton, hemp, wool, and rayon. Suitable synthetic fibers include fibers commonly used in textiles, including but not limited to polyester and polypropylene fibers.

Various forming methods can be used to form a suitable fibrous planar substrate, alternatively referred to as a web. For instance, the web can be made by nonwoven dry forming techniques, such as air-laying, or alternatively by wet laying, such as on a papermaking machine. Other nonwoven manufacturing techniques, including but not limited to techniques such as melt blown, spunbonded, needle punched, and hydroentanglement methods may also be used.

The elongate operative members may comprise yarns including any of the following types but not limited to multifilament, bulk continuous filament, heat set, twisted, wrapped, multi-fiber such as thread, single or monofilament, and bicomponent or multi-component. Further, the individual filaments or fibers forming such yarns may be of any cross section, such as round, trilobal, pentalobal, rectangular, star shaped, hollow or irregular, etc. The yarns may be composed of a wide variety of chemistries including acrylic, polyester, nylon, cotton, rayon, glass, polypropylene, polyethylene, odor absorbent material known in the art, absorbent gelling material or combinations thereof. Also, the yarns may exhibit a coloration which is distinctly contrasting from the substrate in order to provide a decorative attribute.

The elongate operative members may alternatively comprise any type of tapes. The tapes may be made from films, wovens, nonwovens, cellulose, etc. Tapes are generally defined by their cross section which is substantially wide relative to the thickness. The cross section of the tapes may have a variety of shapes not limited to those such as ribbons, wavy tapes, or a variety of geometric shapes attached by filaments such as strings or the like. Like the yarns, the tapes may be composed of a wide variety of chemistries including acrylic, polyester, nylon, cotton, rayon, glass, polypropylene, polyethylene, odor absorbent material known in the art, absorbent gelling material or composites thereof. Like the yarns, the tapes may also exhibit a coloration which is distinctly contrasting from the substrate in order to provide a decorative attribute.

The serviceable operations performed by the elongate operative members can be directed toward a wide variety of user beneficial outcomes including but not limited to: cleaning, bleaching, staining, coloring, sanitizing, polishing, deodorizing, disinfecting, medicating, drugging, chemically etching, surface modifying, smoothing, dyeing, de-gumming, coating, encapsulating, picking up materials, chemically reacting, sealing, lightening, darkening, frosting, greasing, thickening, gelling, painting, inking, printing, scenting, saturating, drying, absorbing, adsorbing, detoxifying, anti-static, de-magnifying, magnifying, tackifying, de-linting, plating, purifying, basting, wiping, chemically reacting including oxidizing, reducing and neutralizing, solidifying, liquefying, hardening, softening, tanning, treating, moisturizing, lotioning, salting, thinning, styling, preserving, lubricating, cleansing, and dissolving.

Some specific exemplary serviceable operations for the elongate operative members include dispersing or delivering an active agent wherein the function of the active agent is to clean, condition, color, modify, chemically react with, catalyze a chemical reaction with, sanitize, disinfect, deodorize, sterilize, kill (such as insects, microorganisms), coat, modify pH, modify biological activity, inhibit, or protect an object. Accordingly, the active agents include, but are not limited to, deodorants, enzyme inhibitors, anti-microbials, anti-fungals, skin care compositions, cleaning agents, surface modification agents, or pH control agents.

As previously defined, a stimulus influences the activity of an object (i.e. the elongate operative member). Such stimulus may comprise electrical, chemical, biological, biochemical, physical, or mechanical properties. Examples of stimuli include, but are not limited to, attitude, pressure, temperature, flow rates, motion, moisture, enzymes, bacteria, pH, conductivity, resistance, capacitance, inductance, electrical and/or electromagnetic fields, light, presence of a material or substance or change in the condition of a material or substance, other properties or events (e.g., occurrence or presence of a material) and combinations thereof. In order to react to such stimuli, the elongate operative members of the present invention may comprise a responsive system including a sensor capable of detecting the presence of a material (such as but not limited to a pathogen, a fluid, or a bodily waste) by sensing a component of the material. Examples of articles having such responsive systems are described in copending application Ser. No. 09/342,785 titled “Disposable Treatment Article having a Responsive System,” which is incorporated herein by reference.

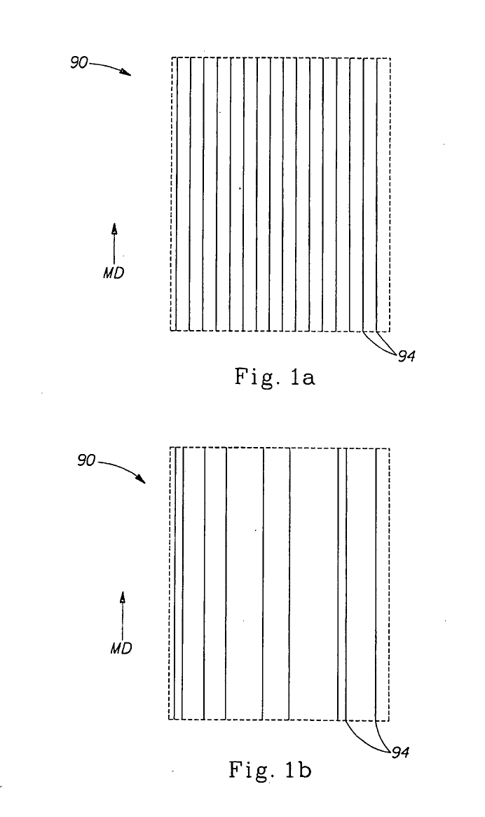

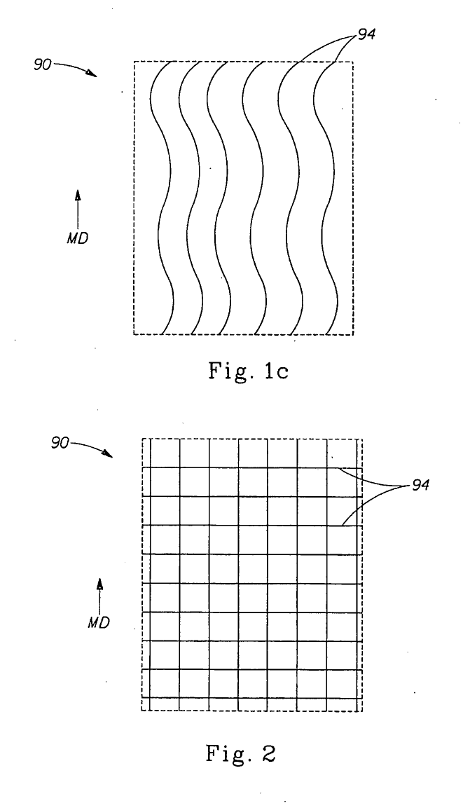

For composite fabric panels 90 of the present invention, the elongate operative members 94 may be disposed unidirectionally along either a longitudinal or transverse axes of the substrate or diagonally to such longitudinal and transverse axis. In such unidirectional arrangements, the elongate operative members 94 can extend along parallel, evenly spaced paths as shown in FIG. 1a or unevenly spaced paths as shown in FIG. 1b. The elongate operative members 94 can also be arranged unidirectionally in nonlinear (such as sinusoidal) paths as depicted in FIG. 1c. Alternatively, the elongate operative members 94 may be disposed bi-directionally with respect to the panel forming an intersecting pattern as shown in FIG. 2. Further, the elongate operative members 94 may be disposed multi-directionally with respect to the substrate, arranged in a random configuration.

The elongate operative members 94 can be joined (via adhesive, weld etc.) to a single layer substrate comprising a unitary planar body. Alternatively, the elongate operative member can be embedded within the unitary body of the substrate. In one embodiment wherein the substrate comprises a nonwoven, the elongate operative members 94 may be embedded within the fibers of the nonwoven. For multiple ply substrates, the elongate operative members 94 may be interposed between two plies and joined to one or both plies or not joined to either ply.

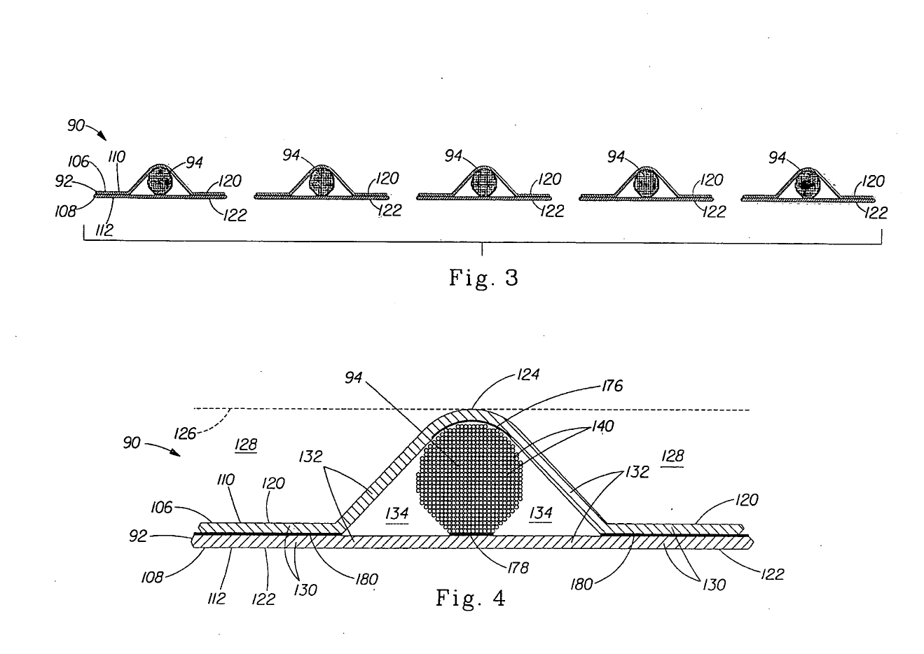

In one exemplary composite fabric panel 90 depicted in FIGS. 3 and 4, the substrate comprises a multiply nonwoven sheet wherein the elongate operative members 94 are disposed in a parallel arrangement between two layers of nonwoven. The elongate operative members delineate sections of the layers between two adjacent elongate operative members. In this arrangement, the sections 120 and 122 of the layers 106 and 108 are joined together by bonds 180 at locations spaced transversely from the elongate operative members 94. In the embodiment shown in FIG. 4, the elongate operative members are also joined to the layers 106 and 108 through bonds 176 and 178. The sections 120 and 122 of the layers 106 and 108 thus have adjoining portions 130 that are spaced transversely from the elongate operative members 94, and further have spaced-apart portions 132 that converge transversely from the elongate operative members 94 to the adjoining portions 130. The elongate operative members 94 and the spaced-apart portions 132 of the layers 106 and 108 define channels 134 that extend alongside the elongate operative members 94.

Liquid impinging upon the sheet 92 can accumulate in the channels 134. When the accumulated liquid contacts the elongate operative members 94, the elongate operative members 94 can be made to perform a dispersing function by drawing the liquid along their lengths. In accordance with this feature of the invention, the elongate operative members 94 extend in the machine direction MD (FIG. 1) of the nonwoven fabric sheet 92. This enables liquid impinging upon the composite fabric sheet to be dispersed from the point of contact to the edges of the panel through the channels 134 extending alongside the elongate operative members 94. Further, the lengthwise orientation of the elongate operative members 94 can help to block the passage of liquid transversely across the elongate operative members.

For this embodiment, the elongate operative members preferably comprise yarns. Each yarn 94 may have either a monofilament or multifilament structure. As shown in FIGS. 3 and 4, the yarns 94 preferably have multifilament structures. This enables the yarns 94 to disperse liquid from the point of contact to the end edges by capillary action in addition to transporting the liquid along their peripheral surfaces. Hydrophilic materials may be included in or on the yarns to enhance the liquid dispersion achieved by the yarns 94. The dispersing effect of the yarns 94 may also be further enhanced by the addition of materials that have a low capacity for absorbing liquid. Such materials will hasten saturation of the yarns 94 fully along their lengths so that the wetted yarns 94 more quickly and fully disperse the liquid from the point of contact to the end edges.

In addition to absorbing and dispersing liquid, the elongate operative members can be made to perform some of the other aforementioned serviceable operations enhancing the properties of the composite fabric panel. For example, the elongate operative members may detect a specific enzyme in a contaminant and release an enzyme inhibitor in response that acts upon the enzyme detected in the contaminant. Alternatively, the elongate operative members may detect a liquid contaminant and release a compressed foam or absorbent material in response that draws the moisture into the material as it expands. The elongate operative members may also detect the presence of a concentrated chemical environment, such as a concentration of a volatile gas that produces an offensive odor, and release a deodorant in response that eliminates the odor of that volatile gas. Further, the elongate operative members can be made to expand or contract in response to a stimulus in order to perform a mechanical operation or change color indicating the presence of a particular contaminant.

In addition, in response to a stimulus, the elongate operative members may be made to release an active agent serving as a protective barrier. Such protective barriers include silicon oils as disclosed in U.S. Pat. No. 5,648,083 issued Jul. 15, 1997, titled “Personal care Compositions and Wipe Products Containing the Compositions” the disclosure of which is incorporated herein by reference. For this embodiment, the composite fabric panel may comprise a wipe having elongate operative members containing protective barrier agents capable of being released in response to the following stimuli, not limited to, change in temperature, wetting or deformation of the elongate operative members induced by shear, tension or compression.

In one embodiment, the elongate operative members may comprise compression devices comprising an elastic foam having suitable compression and recovery properties so that it is capable of being compressed and held in a compressed state by a soluble film that may dissolve in contact with a contaminant such as water, urine, fecal enzymes, bacteria etc. The compression devices can be made to releases a material (e.g., an active agent) for treating an object, wherein the active agent is stored in or on the compression material. Upon activation of the compression material the active material is released in order for it to perform its intended function. This type of elongate operative member can be referred to as a compression-active material release device. The soluble film may, for example, comprise a plastic film that is soluble to water such as a PVA film supplied by Chris-Craft Industrial Products, Inc. of South Holland, Ill. as MONOSOL M7031 film, or H. B. Fuller Company of St. Paul, Minn. as HL 1636 or HL 1669-X. The film thickness, for example, may also be modified to provide a desired activation. The film used may, for example, have a thickness in the range from about 0.0005 to about 0.0015 inches. An HL 1636 film having a thickness of about 0.001 inches, for example, will activate with a moisture content of about 0.049 grams per square inch.

The soluble film described above may comprise a pH sensitive, water soluble material that forms an envelope around a pH buffer system. Such soluble material may have a pH threshold. The “pH threshold” of a soluble material is the pH at which the material changes from soluble to insoluble or vice-versa. For example, the soluble material may be substantially insoluble at pH of less than 6, but soluble at a pH of greater than 6. Thus, the pH threshold of that material is a pH of 6. In preferred embodiments of the present invention, the pH threshold of the soluble material is preferably between about 5 and about 9, and more preferably between about 5.5 and about 8.5, although other pH thresholds are contemplated. The change in pH may be the cause or trigger for the dissolution of the soluble material. The pH sensitive film may have, for example, a pH threshold in the range of about 5 to 7. The pH buffer, for example, may be a pH 7 phosphate buffer available from Corning, Inc., Corning, N.Y. (Cat #473650). When the threshold pH is reached, the pH buffer is released and functions in a continuous manner via a stoichiometric chemical reaction.

In another embodiment of the present invention, the elongate operative members may comprise a foam as previously described or another resilient material such as a resilient yarn that is twisted creating torsional mechanical potential energy and enclosed in a soluble film envelope as described above. Preferably, the twisted resilient material is held in the twisted position in the soluble film under vacuum. In this embodiment, when a threshold level of moisture, pH, etc. is detected the film or capsule dissolves, discontinuously releasing the vacuum, and releasing the foam. The stored torsional mechanical potential energy causes the foam to unwind and may perform a serviceable operation such as storing, capturing or entrapping contaminants, wiping the surface of an object, applying an active agent to an object, etc.

In yet another embodiment, a pH control agent may be embedded in a film or granules, or held under a film of a pH-sensitive material that is insoluble, i.e., a solid, below a predefined pH (e.g., less than a pH of about 6.0), but soluble above that pH level. Upon detection of the threshold pH level or above, the pH-sensitive embedding or overlying material dissolves, releasing the pH control agent to treat the intended object. In the case of the embedded pH control agent, the elongate operative members release the agent in a continuous manner as the embedding material dissolves. In the case of the pH control agent being held under a film, the elongate operative members release the agent in a discontinuous manner after the film has dissolved. A pH control agent may be a buffer, a pH decreasing agent, e.g., an acid, or a pH increasing agent, e.g., a base. A variation of this embodiment may include a substance that will result in a pH change upon hydrolysis by one or more target enzymes or other component that may be present in a contaminant. When the target enzyme or other component reacts with the substance, the reaction creates a pH change that may react with a pH sensitive material similar to the one described above to release a pH control agent. An enzyme inhibitor may also be embedded in the pH-sensitive material. Presence of the target enzyme, e.g., a fecal enzyme, may result in the conversion of the substance and a change in pH, resulting in the dissolution of the pH-sensitive material and release of the enzyme inhibitor to treat the feces or other object, such as the user's skin or other surface. Exemplary pH sensitive materials are known in the art and include polyacrylamides, phthalate derivatives, formalized gelatin, shellac, keratin, cellulose derivatives, e.g., oxidized cellulose, and polyacrylic acid derivatives. Preferred materials include cellulose acetate phthalate, vinyl acetate, polyvinyl acetate phthalate, hydroxy propyl methyl cellulose phthalate and poly methacrylate blended with acrylic acid and acrylic ester copolymers. Other exemplary materials are described in EP612,520 A2 entitled “pH Triggered Osmotic Bursting Delivery Devices,” which is incorporated herein by reference.

A further embodiment of an elongate operative member of the present invention may deliver, i.e., actively transport, an agent to an object to be treated, a contaminant, a wearer, a user, or an article in response to a stimulus. In this embodiment, for example, the elongate operative member may comprise a compressed resilient foam or a closed system liquid transport member that delivers an agent to the object to be treated.

In another embodiment, the elongate operative members may comprise a pH buffer embedded in a pH sensitive material that allows a continuous release of the pH buffer in a continuous dissolution in increased “non-target” pH water. As the moisture having a non-target pH level comes into contact with the pH sensitive material, the material dissolves in a continuous manner, and releases a quantity of the pH buffer, which changes the pH level of the moisture to the target pH level. As increasing quantities of moisture having a non-target pH level contact the pH sensitive material, the material releases an increasing quantity of the pH buffer.

The elongate operative members of the present invention may also release or deliver one or more feces modifying agents (“FMA's”, “viscous bodily waste modifying agents”, “modifying agents” or “agents”), when fecal matter is sensed by the elongate operative members. The FMA is used at an effective amount for modifying the chemical or physical properties of viscous bodily waste, such as feces and menses. This can include hardening the fecal matter, increasing or decreasing the effective viscosity of feces, increasing or decreasing the ease of dewatering the feces, decreasing the stickiness of the feces, decreasing the adhesion characteristics of the feces, or any combination of the above.

An “effective concentration” of an FMA, as used herein, refers to the relative amount of the agent required to have a measurable effect on the viscosity or hardness of the fecal matter. Preferably, a concentration of an FMA of at least about 0.01 weight percent of the feces to be treated is desirable, and more typically between about 0.1 and about 50 weight percent of the feces is available to the feces.

The Feces Modifying Agent of the present invention may include one or more “water liberating” agents capable of separating the liquid portion of the feces (i.e. water) from the solid structure of the feces and/or reducing the degree of “binding” of the feces water to the solid feces components.

Feces Modifying Agents which act to decrease the viscosity of feces as described above include, but are not limited to the following: organic and inorganic flocculants, and the like. Inorganic flocculants include but are not limited to divalent and trivalent metal salts, including but not limited to salts of iron, aluminum, calcium, and sodium and mixtures thereof. It is believed that such salts form hydrolysis products which associate with the charged surfaces of the particulate matter in the feces colloidal structure, resulting in flocculation (i.e., flocculation via any of the mechanisms described above). Some examples include ferrous chloride, ferric chloride, aluminum sulfate, aluminum chloride hydroxide, sodium aluminate, calcium sulfate, poly-aluminum-silicate-sulfate (available from Handy Chemical, Quebec under the trade name PASS), ferrous sulfate, calcium carbonate, and the like.

Organic flocculants include but are not limited to natural substances like albumin, xanthan gum, and guar gum. Synthetic flocculants are generally non-crosslinked, water-soluble molecules or polymers and may include acrylic and acrylamide polymers and their derivatives (in very low concentrations (a few hundredths of a weight percent)), polyvinyl pyrrolidone, poly methacrylates, polyamines, polyethylene oxide, and allylamine polymers. Preferably, these are cationic polymeric species. (Although applicants do not wish to be bound by theory, it is believed that these agents function by associating with the negatively charged regions of the feces particulate fraction and reducing the net inter-particle repulsive charge.) Some of the synthetic flocculants may act to increase the viscosity of aqueous solutions if used in high concentrations and will be discussed below as feces thickening agents. It is also important to note that if some of the organic flocculants are used in too high a concentration their effect may be reversed. Thus, the water may be held more tightly by the feces due to the tendency of these agents to form gels if used in excess of the amount necessary to associate with the charged particulates.

Some crosslinked derivatives of the synthetic organic flocculants (e.g., polyacrylates), or derivatives thereof, are known in the art as superabsorbent polymers, and function to form water-insoluble gels upon contact with very low viscosity aqueous wastes such as urine and menses. However, because these crosslinked species cannot readily dissociate (i.e., dissolve) and adsorb to the particulate species within the feces matrix, they do not function as flocculants.

Feces Modifying Agents which act to decrease the viscosity of feces as described above may also include reducing agents. For example, agents that reduce disulfide bonds (—S—S-bonds) as found in colonic mucous colomin mucous generally comprises (macromolecular glycoproteins linked by disulfide bonds) can effect a significant viscosity reduction in feces having high mucous content. While not wishing to be bound by theory, it is believed that reduction of the mucin disulfide bonds (which function as crosslinks between mucin polymer chains) significantly reduces the average molecular weight of the glycoprotein structure in feces such as runny feces to a level well below the “gel point” of the mucin (i.e., long-distance structure becomes impossible due to the relatively small size of the glycoproteins). Exemplary reducing agents include sulfites such as sodium hydrogensulphite, sodium sulfite and sodium dithionite, thiols and thiol alcohols (e.g., 2-mercaptoethanol, dithiothreitol, and dithioerythritol), mercaptoacetic acid, sodium thioglycolate, thiolactic acid, thioglycoamide, glycerol monothioglycolate, borohydrides (e.g., sodium borohydride), ternary amines, thiocyanates such as sodium thiocyanate, thiosulfates such as sodium thiosulfate, cyanides such as sodium cyanide, thiophosphates such as sodium thiophosphate, arsenites such as sodium arsenite, phosphines such as triphenyl phosphine, phenols such as thiophenol and p-nitrophenol, betaines, and others including, but not limited to, lithium aluminumhydride, aluminum chloride, guanidine hydrochloride, stannous chloride, hydroxylamine, and LiHB(C₂H₅)₃.

In other preferred embodiments of the present invention, modifying agents which generally increase the structure of the feces by increasing the degree of water binding are employed to increase the viscosity and reduce the mobility of the feces. This may be accomplished via the use of thickening agents in the appropriate concentrations. Thickening agents may be natural or synthetic and are generally water-soluble, (typically non-crosslinked) polymers, such as CMC (carboxymethyl cellulose), hydroxypropyl methylcellulose, hydroxyethyl cellulose, polyacrylic acid and its derivatives, carageenan, polyacrylamide and its derivatives, (polyethylene)imines, gums (such as xanthan, guar, karaya, agar, locust bean gum, pectin, and gum ghatti, or mixtures thereof) and other similar materials. Cationic polymers are preferred due to the anionic surfaces of fecal bacteria and biopolymers. Thickening agents increase the viscosity of the feces by dissolving in the free water in the feces and osmotically “binding” water, thereby increasing the solid “structure” of the feces. Generally, large, insoluble polyelectrolytic polymeric particles such as conventional superabsorbents are not able to dissolve in the feces free water and create a matrix within the feces at the molecular level. Some FMAs may perform differently on different types of feces (e.g., a FMA that acts as a flocculant on one type of feces, may act as a thickening agent on another type due to variance in the structural character of the specific type of feces). One example of this is calcium hydroxide which functions as a flocculant for a runny fecal analog, but as a thickener for a pasty fecal analog in the same concentrations.

In still other preferred embodiments, the modifying agent comprises an ionic complexing agent. Ionic complexing agents may include any single component which complexes with itself or water or other chemical entities in the feces to form regions of increased structure and rigidity within the feces. The resultant complex acts to stabilize or bind water more tightly in the feces. Exemplary ionic complexing agents include ZnO, MgO, MnO, CaO, calcium hydroxide, Al₂O₃, aluminum salts, zinc salts such as zinc acetate and zinc glucanate, gelatin, quaternary ammonium salts, ethanolamines, alginic acid, cetyl trimethyl ammonium bromide and the like). Alternatively, the ionic complexing agent may comprise a two (or more) component system, wherein the complex (i.e., longer-range structure) is created by the interaction of the two added components (e.g., aluminum, calcium, or zinc salts plus alginic acid and/or salts thereof). The ionic complexing agents may form crystal hydrates when complexing with water. In general, calcium-containing compounds or systems (e.g., CaO, calcium hydroxide, and calcium alginate, etc.) are some of the most effective feces modifying agents.

In yet another embodiment, one or more enzymes or microorganisms may be detected by an enzyme-degradable film or capsule, covering the elongate operative members to release an enzyme inhibitor to treat the skin. Exemplary enzyme inhibitors are disclosed in U.S. patent application Ser. No. 09/041,266 entitled “Disposable Absorbent Article Having A Skin Care Composition Containing An Enzyme Inhibitor” filed on Mar. 12, 1998, which is incorporated by reference herein.

Exemplary skin care compositions (or lotions), are disclosed in U.S. Pat. Nos. 5,607,760 entitled “Disposable Absorbent Article Having A Lotioned Topsheet Containing An Emollient And A Polyol Polyester Immobilizing Agent,” issued to Donald C. Roe on Mar. 4, 1997; 5,609,587 entitled “Diaper Having A Lotioned Topsheet Comprising A Liquid Polyol Polyester Emollient And An Immobilizing Agent,” issued to Donald C. Roe on Mar. 11, 1997; 5,635,191 entitled “Diaper Having A Lotioned Topsheet Containing A Polysiloxane Emollient,” issued to Donald C. Roe et al. on Jun. 3, 1997; and 5,643,588 entitled “Diaper Having A Lotioned Topsheet” issued to Donald C. Roe et al. on Jul. 1, 1997, as well as U.S. patent application Ser. Nos. 08/926,532 and 08/926,533, each filed on Sep. 10, 1997, each of the above listed patents and applications are incorporated herein by reference.

As previously described, the composite fabric panel 90 of the present invention may comprise a multi-ply substrate comprising overlapping layers of nonwoven sheets with elongate operative members disposed therebetween. A method of constructing the composite fabric panel 90 includes the use of a pair of rollers 160 and 162, as shown schematically in FIG. 5. The lower layer 108 of the nonwoven fabric sheet 92 is first laid out on a conveyor 164 as a unitary planar body of unbonded fibers. The unbonded fibers are previously mixed with a suitable cementing medium, as known in the art. Elongate operative members comprising yarns 94 are oriented to extend in the machine direction MD and are laid upon the lower layer 108 in the array described above. The upper layer 106 is then deposited over the yarns 94 and the lower layer 108 in the form of a unitary planar body of unbonded fibers and cementing medium. The overlapping components of the panel 90 are then passed through the nip 165 between the rollers 160 and 162. The nonwoven fibers in each layer 106 and 108 become bonded together, and the yarns 94 and layers 106 and 108 become bonded to each other, under the influence of heat and compression developed in the nip 165.

As further shown in FIG. 5, one of the rollers 160 has grooves 170. The grooves 170 extend circumferentially around at least a portion of the cylindrical surface 172 of the roller 160, and are arranged in an array corresponding to the array of yarns 94 so that each yarn 94 passes through a corresponding groove 170 upon moving through the nip 165 between the rollers 160 and 162. The grooves 170 provide clearance to avoid excessive compression of the yarns 94 at the nip 165.

More specifically, the diameter of each yarn 94 is greater than the spacing between the cylindrical surfaces 172 and 174 of the rollers 160 and 162 at the nip 165. The depths of the grooves 170 are specified with reference to the diameters of the yarns 94, and with reference to the spacing at the nip 165, to provide clearance for the yarns 94 to pass through the nip 165 without being compressed to flattened conditions from which they cannot rebound nearly or completely to their original, unflattened conditions, as shown for example in FIG. 4. The depths of the grooves 170 are further specified with reference to the thicknesses of the layers 106 and 108 so that the layers 106 and 108 will be pressed against the yarns 94 sufficiently to form bonds 176 and 178 (FIG. 4) with the yarns 94.

The widths of the grooves 170 also are specified with reference to the diameters of the yarns 94 and the thicknesses of the overlapping layers 106 and 108. With further reference to FIG. 4, the elongated sections 120 and 122 of the layers 106 and 108 are joined together by bonds 180 between the adjoining portions 130 of the sections 120 and 122. The means for producing the bonds 180 include, but are not limited to, adhesives, ultrasonics, and high pressure embossing. (Bonding via high pressure embossing is disclosed in U.S. Pat. No. 3,323,983 issued Sep. 8, 1964 to Palmer and is incorporated herein by reference). The bonds 180 are formed where the adjoining portions 130 are pressed together by the cylindrical roller surfaces 172 and 174. The widths of the grooves 170 exceed the diameters of the yarns 94 so that the converging portions 132 of the layers 106 and 108 pass through the grooves 170 with the yarns 94. This ensures that the converging portions 132 of the layers 106 and 108 will not be pressed and bonded together by the cylindrical roller surfaces 172 and 174.

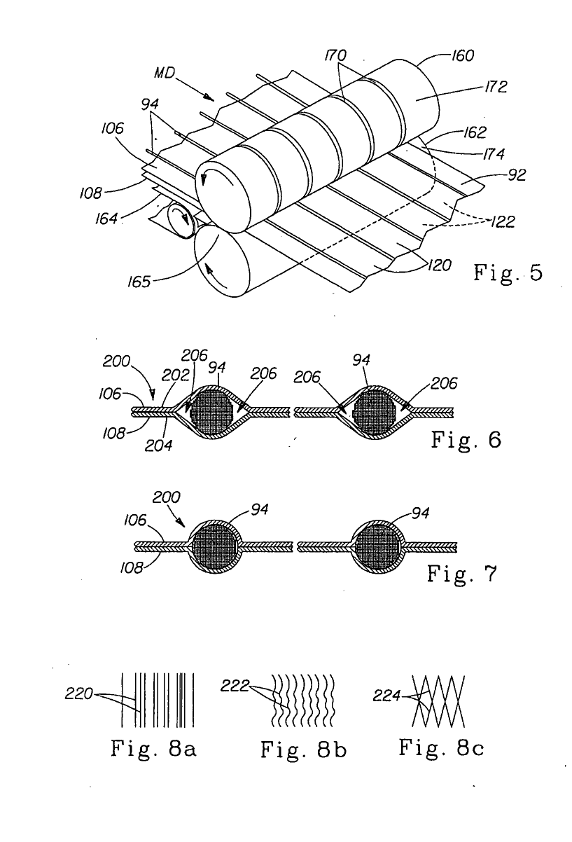

The lower roller 162 also may have grooves for guiding the yarns 94 through the nip 165. Such an embodiment can be used to form a composite fabric panel 200 (FIG. 6) having a ribbed feel at each of its opposite side surfaces 202 and 204. The grooves in the rollers 160 and 162 could be sized for the formation of channels 206 alongside the yarns 94, as shown in FIG. 6, or to press the layers 106 and 108 together fully between the yarns 94 without the formation of channels, as shown in FIG. 7. Additionally, a composite fabric panel constructed in accordance with the invention may have arrays of yarns that differ from the parallel, evenly spaced-apart array of yarns 94. Such arrays could include yarns 220 that are unevenly spaced apart (FIG. 8A); yarns 222 that extend along sinusoidal or otherwise nonlinear paths (FIG. 8B); and/or yarns 224 that intersect (FIG. 8C). Corresponding arrays of roller grooves would be used to control compression of the yarns and the formation of channels alongside such yarns in substantially the same manner as described above.

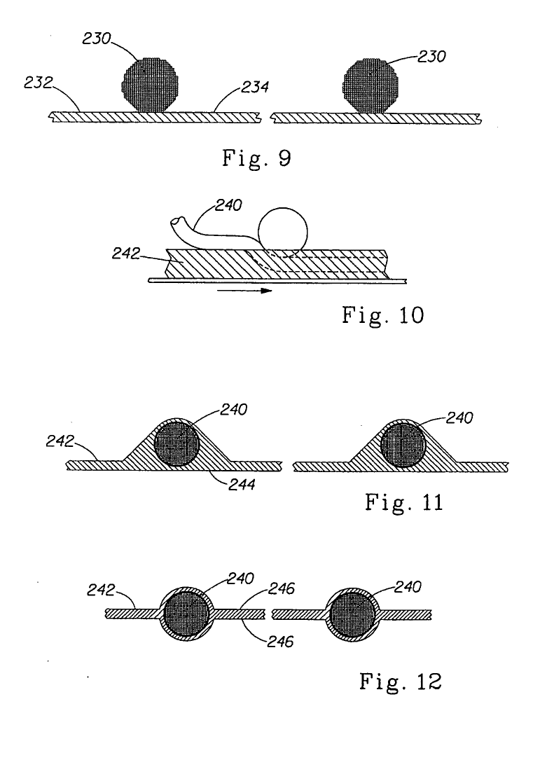

The composite fabric panel of the present invention may also comprise a unitary generally planar body of nonwoven fibers with the elongate operative members embedded within the body of fibers. For this embodiment, the composite fabric panel may be made by engaging yarns directly with a nonwoven fabric sheet. For example, as shown in FIG. 9, yarns 230 are engaged with a nonwoven fabric sheet 232 by mounting the yarns 230 on a side surface 234 of the sheet 232. As shown in FIG. 10, yarns 240 are embedded within a sheet 242 of nonwoven fibers when the sheet 242 is in an unbonded (or partially bonded) noncohesive condition suitable for receiving the yarns 240 in this manner. The noncohesive sheet 242 and the embedded yarns 240 are then passed through a nip between a pair of rollers to form a bonded composite fabric panel in accordance with the invention. Such rollers may be configured with grooves that provide the nonwoven fabric sheet 242 with a planar side surface 244 extending across the yarns 240, as shown in FIG. 11, or with grooves that provide undulating opposite side surfaces 246 extending across the yarns 240, as shown in FIG. 12.

### Exemplary Embodiment

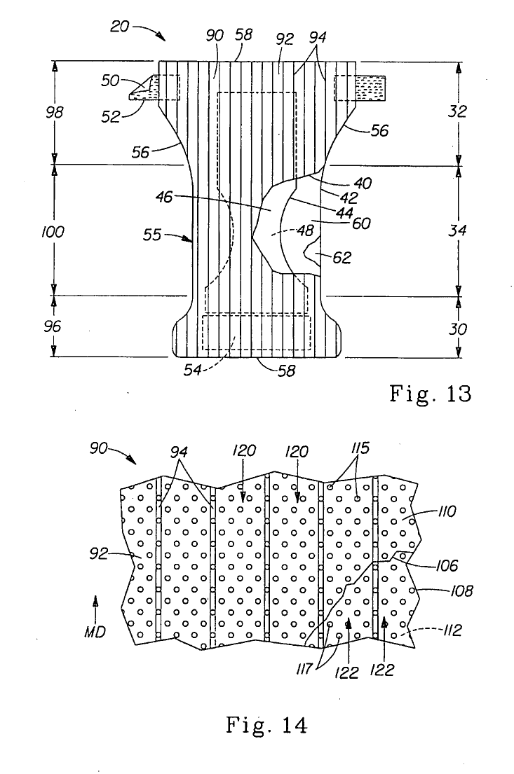

Although the composite fabric panel of the present invention is equally applicable to the several types of absorbent articles previously mentioned, a preferred embodiment of an absorbent article of the present invention is a unitary disposable absorbent article, such as the disposable diaper 20, shown in FIG. 1. FIG. 1 is a plan view of the diaper 20 in its flat out, uncontracted state (i.e., without elastic induced contraction) with portions of the structure being cut away to more clearly show the underlying structure of the diaper 20 and with the portion of the diaper 20 which contacts the wearer facing the viewer. The diaper 20 is shown in FIG. 13 to have an elongated configuration. One end portion 30 of the diaper 20 is configured as a front waist region of the diaper 20. The opposite end portion 32 is configured as a rear waist region of the diaper 20. An intermediate portion 34 of the diaper 20 is configured as a crotch region which extends longitudinally between the front and rear waist regions 30 and 32. The waist regions 30 and 32 generally comprise those portions of the diaper 20 which, when worn, encircle the waist of the wearer. The waist regions 30 and 32 may include elastic elements such that they gather about the waist of the wearer to provide improved fit and containment. The crotch region 34 is that portion of the diaper 20 which, when the diaper 20 is worn, is generally positioned between the legs of the wearer.

The diaper 20 preferably comprises a liquid pervious topsheet 40, a liquid impervious backsheet 42, and an absorbent core 44 encased between the topsheet 40 and the backsheet 42. The absorbent core 44 preferably has a body surface 46 which generally faces the wearer and a garment surface 48 which generally faces away from the wearer. The diaper 20 preferably also includes a fastener such as a hook and loop type fastening system 50 including at least one engaging component 52 (male fastening component) and at least one landing zone 54 (female fastening component). The diaper 20 may also include such other features as are known in the art including leg cuffs, front and rear ear panels, waist cap features, elastics and the like to provide better fit, containment and aesthetic characteristics. Such additional features are well known in the art and are described in U.S. Pat. No. 3,860,003; and U.S. Pat. No. 5,151,092 which are incorporated by reference herein.

The composite fabric panel of the present invention may be suitable for other diaper embodiments including those disclosed in U.S. Pat. No. 6,010,491 issued Jan. 4, 2000; U.S. Pat. No. 5,873,870 issued Feb. 23, 1999; U.S. Pat. No. 5,897,545 issued Apr. 27, 1999; U.S. Pat. No. 5,904,673 issued May 18, 1999; U.S. Pat. No. 5,931,827 issued Aug. 3, 1999; U.S. Pat. No. 5,977,430 issued Nov. 2, 1999 and U.S. Pat. No. 6,004,306 issued Dec. 21, 1999, the disclosures of which are incorporated herein by reference.

The topsheet 40 in the first preferred embodiment of the invention is defined by a composite fabric panel 90. As best shown in FIG. 14, the composite fabric panel 90 includes a nonwoven fabric sheet 92 and plurality of elongate operative members composed of yarns 94 that are engaged directly with the nonwoven fabric sheet 92. The periphery of the nonwoven fabric sheet 92 corresponds to the periphery of the topsheet 40 and, in turn, the periphery 55 of the diaper 20 (FIG. 13). The nonwoven fabric sheet 92 thus has an elongated configuration with one end portion 96 configured as a front waist region of the sheet 92, an opposite end portion 98 configured as a rear waist region of the sheet 92, and an intermediate portion 100 configured as a crotch region of the sheet 92. Those regions 96, 98 and 100 of the nonwoven fabric sheet 92 are preferably coextensive with the corresponding regions 30, 32 and 34 of the diaper 20 itself.

In this particular embodiment of the invention, the nonwoven fabric sheet 92 has a laminate structure including a pair of overlapping layers 106 and 108 (FIG. 14) of nonwoven fabric that are bonded together. The upper layer 106 has an outer surface 110 that contacts the wearer's skin. The lower layer 108 has an outer surface 112 that overlies the absorbent core 44 and the backsheet 42. The yarns 94 are enclosed within the nonwoven fabric sheet 92 between the overlapping layers 106 and 108.

The nonwoven fabric sheet 92 has a machine direction MD indicated by the arrow shown in FIG. 14. The layers 106 and 108 are preferably alike, and each is formed of nonwoven fibers that are bonded together in random orientations relative to each other. Such fibers may be formed of any material that provides the nonwoven fabric sheet 92 with material properties suitable for the diaper topsheet 40. Preferably, the nonwoven fibers are formed of hydrophobic material to isolate the wearer's skin from liquids in the absorbent core 44. If the nonwoven fabric sheet 92 is thus formed of hydrophobic material, at least the outer surface 110 of the upper layer 106 may be treated to be hydrophilic so that liquids will transfer through the topsheet 40 more rapidly. This diminishes the likelihood that body exudates will flow off the topsheet 40 rather than being drawn through the topsheet 40 and being absorbed by the absorbent core 44. The topsheet 40 can be rendered partially hydrophilic in this manner by treating the nonwoven fabric sheet 92 with a surfactant including spraying the sheet 92 or the outer layer 106 with a surfactant or immersing it into the surfactant. A more detailed discussion of such a treatment is contained in U.S. Pat. Nos. 4,988,344 entitled “Absorbent Articles with Multiple Layer Absorbent Layers” issued to Reising et al., on Jan. 29, 1991, and U.S. Pat. No. 4,988,345 entitled “Absorbent Articles with Rapid Acquiring Absorbent Cores” issued to Reising on Jan. 29, 1991, each of which is incorporated herein by reference.

Suitable structural properties of the topsheet 40, such as permeability and breathability, can be provided by interstices between the bonded fibers in the layers 106 and 108 of the nonwoven fabric sheet 92. Such interstices can be formed in a known manner. A preferred structural property, as indicated in FIGS. 13 and 14, is a significant degree of transparency such that the enclosed yarns 94 are distinctly visible through the upper layer 106 of the nonwoven fabric sheet 92. In accordance with this feature of the invention, the sheet 92 preferably has a first coloration and the yarns 94 preferably have a second, distinctly contrasting coloration. As further shown in FIG. 14, the permeability and breathability of the sheet 92 are enhanced by aligned apertures 115 and 117 in the layers 106 and 108, respectively. The apertures 115 and 117 are formed in a known manner and are preferably provided in an array extending uniformly throughout the entire sheet 92.

The yarns 94 may be configured as rib members to provide the sheet 92 with a ribbed feel. Specifically, the yarns 94 are received between the layers 106 and 108 of the sheet 92 in spaced-apart positions in which they delineate elongated sections 120 and 122 of the layers 106 and 108 that extend transversely between pairs of adjacent yarns 94. The yarns 94 in this embodiment are alike, and are parallel and evenly spaced-apart such that the elongated sections 120 and 122 of the layers 106 and 108 have uniform, equal widths. The width of each section 120 and 122 is preferably several times greater than the diameter of each yarn 94. However, the diameters of the yarns 94 are great enough to impart an undulating contour, and thereby to impart a ribbed feel, to the outer surface 110.

The yarns 94 are further sized to provide a standoff feature of the composite fabric panel 90, as indicated in FIG. 4. In accordance with this feature of the invention, the undulating contour of the outer side surface 110 defines crests 124 at which the panel 90 contacts the wearer's skin 126 (shown schematically), and defines intervening troughs 128 at which the outer surface 110 is spaced from the wearer's skin 126. This is helpful for comfort, and also to inhibit the formation of imprints or other blemishes on the skin 126 where the circular edges of the apertures 115 (FIG. 14) might otherwise contact the skin 126.

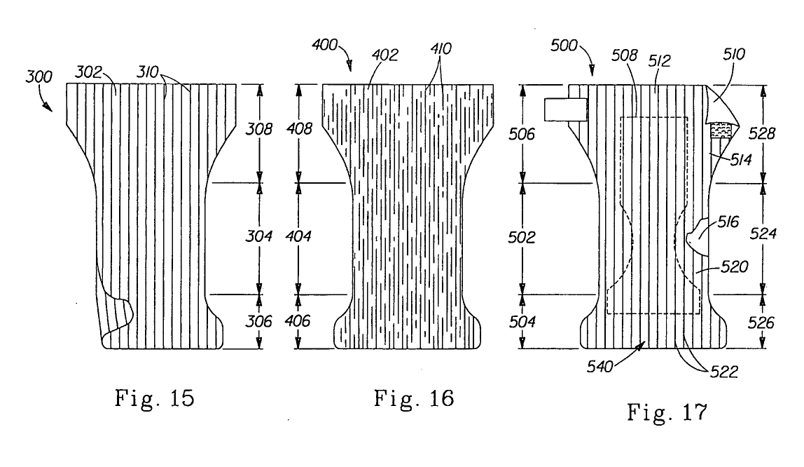

Another composite fabric panel 300 is shown in FIG. 15. Like the panel 90 shown in FIG. 13, the panel 300 (as well as the other panels described above) is configured as a diaper topsheet. The panel 300 thus includes a nonwoven fabric sheet 302 having an elongated configuration with a crotch region 304 between a pair of waist regions 306 and 308. A plurality of yarns 310 are engaged directly with the sheet 302, and are configured as rib members that extend lengthwise of the sheet 302 through the crotch region 304 and the waist regions 306 and 308.

Unlike the yarns 104 in the panel 90, the yarns 310 in the panel 300 comprise filaments that are formed of heat shrinkable material. When the panel 300 is being constructed, the yarns 310 are heated sufficiently for the heat shrinkable material to shrink. This causes the yarns 310 to contract longitudinally and thereby to deflect the sheet 302 so as to impart an undulating surface contour to the sheet 302. The diaper topsheet defined by the panel 300 is thus provided with a rippled configuration for a cushioning effect, as indicated schematically.

A similar rippled configuration is provided for the alternative topsheet 400 shown in FIG. 16. This topsheet 400 includes a nonwoven fabric sheet 402 with an elongated configuration defining a crotch region 404 and a pair of waist regions 406 and 408. However, this topsheet 400 does not include yarns configured as longitudinally extending rib members. Instead, this topsheet 400 includes an array of separate filaments 410 that are bonded to the sheet 402. The filaments 410 are formed of heat shrinkable material, and have shortened conditions attained upon shrinkage of that material under the influence of heat. The sheet 402 thus has an undulating, rippled surface contour as a result of deflection imparted to the sheet 402 by the filaments 410 upon shrinkage of the heat shrinkable material.

Another example of an absorbent article comprising a preferred embodiment of the present invention, namely a disposable diaper 500, is shown in FIG. 17. (The diaper 500 is viewed from the back in FIG. 17, whereas the diaper 20 is viewed from the front in FIG. 13.) Like the diaper 20 described above, the diaper 500 has an elongated configuration with a crotch region 502 located between front and rear waist regions 504 and 506, respectively. The diaper 500 also includes an absorbent core 508 encased between a topsheet 510 and a backsheet 512. However, in this embodiment of the invention the backsheet 512, rather than the topsheet 510, includes a composite fabric panel 514 constructed in accordance with the invention.

In addition to the composite fabric panel 514, the backsheet 512 further includes an inner web 516 that renders the backsheet 512 impervious to liquid. The inner web 516 may have any suitable structure known in the art and may be joined to the composite fabric panel 514 in any suitable manner known in the art.

Like each of the composite fabric panels described above, the composite fabric panel 514 includes a nonwoven fabric sheet 520 and a plurality of yarns 522 that are engaged directly with the sheet 520. The sheet 520 has an elongated configuration with a crotch region 524 between front and rear waist regions 526 and 528. Each of those regions 524, 526 and 528 of the nonwoven fabric sheet 520 is preferably coextensive with the corresponding region 502, 504 or 506 of the diaper 500 itself. The yarns 522 are configured as rib members that extend lengthwise of the sheet 520 from the crotch region 524 into at least one of the waist regions 526 or 524, and preferably into and fully through both waist regions 526 and 528.

The composite fabric panel 514 in the diaper 500 may be constructed in accordance with any of the particular features of the invention described above with reference to FIGS. 1a-12. In accordance with another particular feature of the invention, the composite fabric panel 514 may serve as a landing zone 540 corresponding to locations where engaging components 542 of a diaper fastening system are configured to land upon fastening of the diaper 500 on the wearer. The landing zone 540 may be integral and defined by the composite fabric panel 514 and need not include a landing structure that is distinct from the overall structure of the panel 514. The undulating surface provided by the elongate operative members (yarns 522) may extend through any portion of the landing zone 540 to provide an engageable landing surface. The undulating surface and/or the elongate operative members may help restrain movement of the engaging components 542 once engaged with the surface and may provide an increase in the shear or peal strength of the fastener. The panel 514 of the present invention may also be modified by processes known in the art, such as ring-rolling and/or otherwise mechanically or chemically altering all or a portion of the panel. Fastening systems employing engageable fasteners of the type suitable for use in the present invention and processes for modifying the characteristics of the fastening system are disclosed in U.S. Pat. No. 5,735,840 issued Apr. 7, 1998, U.S. Pat. No. 5,928,212 issued Jul. 27, 1999, and U.S. Pat. No. 5,624,427 issued Apr. 29, 1997 the disclosures of which are incorporated herein by reference.

In an alternate embodiment, the composite fabric panel may be limited to a separate distinct portion of the backsheet by attaching a patch of the composite fabric panel to the backsheet at the landing zone. Like the fastening system described above, the elongate operative members may provide an improved engageable landing structure that restrains movement of the engaging components while at the same time provide functional attributes that accentuate the characteristics of the diaper component.

While particular embodiments and/or individual features of the present invention have been illustrated and described, it would be obvious to those skilled in the art that various other changes and modifications can be made without departing from the spirit and scope of the invention. Further, it should be apparent that all combinations of such embodiments and features are possible and can result in preferred executions of the invention. Therefore, the appended claims are intended to cover all such changes and modifications that are within the scope of this invention.

What is claimed is:

1. A composite fabric panel comprising a substrate and a plurality of elongate operative members engaged directly with the substrate, wherein the elongate operative members disperse an active agent in response to a stimulus.

2. A composite fabric panel as defined in claim 1 wherein the substrate includes a pair of overlapping nonwoven fabric layers, and the elongate operative members are enclosed within the substrate between the layers.

3. A composite fabric panel as defined in claim 2 wherein the elongate operative members delineate sections of the layers between pairs of adjacent elongate operative members, the sections of the layers having adjoining portions that are spaced transversely from the elongate operative members, and further having spaced-apart portions that converge transversely from the elongate operative members to the adjoining portions, such that the elongate operative members and the spaced-apart portions of the sections enclose channels that extend alongside the elongate operative members.

4. A composite fabric panel as defined in claim 1 wherein the substrate comprises a unitary generally planar body of nonwoven fibers, and the elongate operative members are embedded within the unitary generally planar body of nonwoven fibers.

5. A composite fabric panel as defined in claim 4 wherein the elongate operative members are bonded to the substrate and comprise filaments formed of heat shrinkable material, the elongate operative members deflecting the substrate upon shrinkage of the filaments under the influence of heat.

6. A composite fabric panel as defined in claim 1 wherein the substrate has a side surface with an undulating contour extending across the elongate operative members and the substrate has an opposite side surface with a planar contour extending across the elongate operative members.

7. A composite fabric panel as defined in claim 1 wherein each elongate operative member comprises at least one multifilament yarn or at least one tape.

8. A composite fabric panel as defined in claim 1 wherein elongate operative members are substantially inelastic.

9. A composite fabric panel as defined in claim 1 wherein the elongate operative members extend along parallel paths.

10. A composite fabric panel as defined in claim 9 wherein the paths are curvilinear.

11. A composite fabric panel as defined in claim 1 wherein the active agent comprises an anti-microbial, an enzyme inhibitor, a pH neutralizer, a protective barrier agent, a feces modifying agent, or a medication.

12. A composite fabric panel as defined in claim 1 wherein the stimulus comprises wetting, a change in temperature, a presence of a concentrated chemical environment, or a presence of an enzyme.

13. An absorbent article comprising a composite fabric panel, the composite fabric panel comprising a substrate and a plurality of elongate operative members engaged directly with the substrate forming an undulating contour across the substrate; the elongate operative members disperse an active agent in response to a stimulus.

14. An absorbent article as defined in claim 13 wherein the absorbent article is a disposable diaper.

15. An absorbent article as defined in claim 13 further comprising a topsheet wherein the topsheet comprises the composite fabric panel.

16. An absorbent article as defined in claim 13 further comprising a backsheet wherein the backsheet comprises the composite fabric panel.

17. An absorbent article as defined in claim 13 further comprising a backsheet and a fastening system wherein the composite fabric panel provides a landing zone separate from the backsheet or a landing zone which is integral with the backsheet for the fastening system.

18. An absorbent article as defined in claim 13 wherein the substrate has a crotch region located between a pair of opposite end regions, the elongate operative members extending from the crotch region into at least one of the end regions.

19. An absorbent article as defined in claim 13 wherein the active agent comprises an anti-microbial, an enzyme inhibitor, a pH neutralizer, a protective barrier agent, a feces modifying agent, or a medication.

20. An absorbent article as defined in claim 13 wherein the stimulus comprises wetting, a change in temperature, a presence of a concentrated chemical environment, or a presence of an enzyme.

21. An absorbent article as defined in claim 13 wherein each elongate operative member comprises at least one multifilament yarns or at least at least one tape.

22. An absorbent article as defined in claim 13 wherein elongate operative members are substantially inelastic.

23. An absorbent article as defined in claim 13 wherein the absorbent article is a feminine hygiene article.
